(19) United States
(12) Patent Application Publication
(10) Pub. No.: US 2011/0136880 A1
(43) Pub. Date: Jun. 9, 2011

(54) Latrunculin-Based Macrolides and Their Uses

(76) Inventors: Khalid El Sayed, West Monroe, LA (US); Mohammad Khanfar, Chicago, IL (US)

(21) Appl. No.: 12/960,681

(22) Filed: Dec. 6, 2010

Related U.S. Application Data
(60) Provisional application No. 61/267,575, filed on Dec. 8, 2009.
(60) Provisional application No. 61/417,942, filed on Nov. 30, 2010.

(51) Int. Cl. A61K 31/427; C07D 417/04; A61P 35/04

(52) U.S. Cl. 514/369; 548/187

(57) ABSTRACT

Latrunculin derivatives are disclosed, as are anti-invasive and cytotoxic uses for latrunculins and latrunculin derivatives, and semisyntheses of latrunculin derivatives. The latrunculins and latrunculin derivatives are useful, for example, in treating cancers.

The benefit of the Dec. 8, 2009 filing date of provisional patent application Ser. No. 61/267,575, and of the Nov. 30, 2010 filing date of provisional patent application Ser. No. 61/417,942 are claimed under 35 U.S.C. §119(e).

This invention was made with government support under grant P20RR16456 awarded by the National Institutes of Health. The government has certain rights in the invention.

This invention pertains to latrunculin-based macrolides and their uses, including their uses for anti-invasive and cytotoxic activity, for example against cancer cells.

### BACKGROUND

The microfilament cytoskeleton protein actin plays an important role in cell biology. Actin affects cytokinesis, morphogenesis, and cell migration. Actin forms versatile, dynamic polymers that define cell polarity, control cell shape, promote stable cell-cell and cell-matrix adhesion, organize organelles, control cell shape, and generate the protrusive forces required for migration. These functions can fail and become abnormal in cancer cells.

The cytoskeleton comprises three principal elements: actin microfilaments, microtubules, and intermediate filaments. The microtubules form a polarized network, facilitating organelle and protein movement throughout the cell. The intermediate filaments are rigid components that help maintain overall cell shape. The actin cytoskeleton and proteins involved in actin regulation and function constitute over 25% of the total protein in a typical cell. The cytoskeletal elements regulate a cell's motility, adhesion, migration, exocytosis, endocytosis, and division. Actin filaments can be disrupted in cancer cells. Malignant cells typically alter the polymerization or remodeling of actin, thereby altering cell morphology and phenotype. Actin alterations are progressive. Distinct actin remodeling profiles tend to correlate with different stages of cancer development and progression.

The macrolide latrunculins A and B were originally isolated from the Red Sea sponge Negombata magnifica. Latrunculin A contains a 16-membered ring, and a unique 2-thiazolidinone moiety connected via a tetrahydropyran (THP) ring. Latrunculins A and B and their derivatives have antiangiogenic, antiproliferative, antimicrobial, and anti-metastatic activities. Latrunculins can disrupt microfilament organization without affecting the microtubular system. Latrunculin A (compound 1) reversibly binds the cytoskeleton actin monomers, forming a 1:1 complex with G-actin and disrupting its polymerization. Latrunculin A has high selectivity, rapid onset of action, and remarkable potency, exceeding those of cytochalasin D. Latrunculin A decreases intraocular pressure and increases outflow facility in monkeys, without adverse side effects on the cornea. Latrunculin A has antiviral and antibacterial activities; it inhibits the stress-activated MAP kinase (SAPK) pathway; and it suppresses hypoxia-induced factor (HIF-1) activation in breast cancer cells.

I. Spector et al., “Latrunculins: Novel Marine Toxins That Disrupt Microfilament Organization in Cultured Cells,” Science, 1983, 219, 493-495 discloses the purification of latrunculins A and B from the sponge Latrunculia magnifica, and their toxic effects against cytoskeletal microfilaments.

R. Longley et al., “Evaluation of marine sponge metabolites for cytotoxicity and signal transduction activity,” J Nat Prod, 1993, 56, 95-920; and H. Konishi et al., “Latrunculin A has a strong anticancer effect in a peritoneal dissemination model of human gastric cancer in mice,” Anticancer Res, 2009, 29, 2091-2097 reported the use of Latrunculin A against lung and human gastric cancers, respectively, in mice.

K. A. El Sayed, D. T. A Youssef, D. Marchetti, J. Nat. Prod., 2006, 69, 219-223 reported that the latrunculin derivative N-acetyllatrunculin B displayed anti-migratory activity.

A 100 nM dose of latrunculin A 17-O-phenyl carbamate (compound 18) inhibits the migration of prostate cancer cells PC-3M-CT⁺ in spheroid disaggregation and Matrigel assays, without cytotoxicity. See K. El Sayed et al., Latrunculin A and Its C-17-O-carbamates inhibit prostate tumor cell invasion and HIF-1 activation in breast tumor cells, J. Nat. Prod. 2008, 71, 396-402.

T. Amagata et al., “Interrogating the Bioactive Pharmacophore of the Latrunculin Chemotype by Investigating the Metabolites of Two Taxonomically Unrelated Sponges,” J. Med. Chem. 2008, 51, 7234-7242 discloses latrunculin analogues from the sponges Cacospongia mycofijiensis and Negombata magnifica obtained by reisolation, a repository, new derivatives, and a synthetic analogue. Cytotoxicities were reported against both murine and human cancer cell lines.

A. Fürstner et al., “Latrunculin Analogues with Improved Biological Profiles by ‘Diverted Total Synthesis’: Preparation, Evaluation, and Computational Analysis,” Chem. Eur. J. 2007, 13, 135-149 reported the preparation of latrunculin-like compounds with actin-binding capacity.

I. Spector et al., “New Anti-Actin Drugs in the Study of the Organization and Function of the Actin Cytoskeleton,” Micro. Res. Tech. 1999, 47, 18-37 gives a review of several marine sponge-derived compounds, including latrunculins, and their activities against the actin cytoskeleton.

M. Khanfar et al., “Semisynthetic latrunculin derivatives as inhibitors for metastatic breast cancer: Biological evaluations, preliminary structure-activity relationship, and molecular modeling studies,” ChemMedChem, 2010, 5, 274-285 is a recent paper based upon some of the same work that is disclosed in the present application.

Likewise, see M. Khanfar et al., “3D-QSAR Studies of latrunculin-based actin polymerization inhibitors using CoMFA and CoMSIA approaches,” European J Med. Chem., 2010, 9, 3662-3668.

### SUMMARY

We have discovered novel anti-invasive and cytotoxic uses for latrunculins and latrunculin derivatives. In addition, we have also discovered a number of novel latrunculin derivatives possessing anti-invasive and cytotoxic activity, including activity against cancer cells. In the examples described below, the generic structures, as well as the specific compounds 3-13, 15-19, 23, 24, and 26-34, and their uses are all believed to be novel. Additionally, it is believed that the biological activity of compound 14 described here is novel.

Cancers that may be treated through the present invention include, but are not limited to, prostate, breast, ovary, urothelial, pancreas, glioblastoma, melanoma, ocular melanoma, gastric, and non-small cell lung cancers.

Latrunculins and latrunculin analogs can be totally synthesized. However, due to the cost and complexity of a total synthesis it is preferred to use a natural source as a starting material in a semi-synthesis. Latrunculins and latrunculin analogs occur naturally in a number of sponges and mollusks. See, e.g., T. Amagata et al., “Interrogating the bioactive pharmacophore of the latrunculin chemotype by investigating the metabolites of two taxonomically unrelated sponges,” J. Med. Chem., vol. 51, pp. 7234-7242 (2008), including the Supporting Information. The sponge Negombata magnifica is a preferred source due to its high yield of latrunculin. See Khalifa et al, 2006: J Chrom. B, 2006, 832, 47-51.

In the latrunculin A molecule we have substituted the C-17 hydroxyl and thiazolidinone nitrogen with various aromatic and aliphatic groups. We found that the C-17/C-15 lactol hydroxyl is important for actin-binding affinity, and noticeably affects the antiproliferative and anti-invasive activities, in latrunculins A and B, respectively. Alkyl substitutions on the C-17 hydroxyl diminished biological activity. Aromatic substitutions at the thiazolidinone nitrogen enhanced activity.

Latrunculins 3 and 14 (NSC 751494 and 749377, respectively) showed remarkable antiproliferative activity in vitro, and were selected for future testing in animals. Other latrunculins with reduced actin-binding affinity showed superior anti-migratory and anti-invasive activities without cytotoxicity, and may also have therapeutic potential. N-p-Methoxyphenylacetyl-17-O-methyllatrunculin A (compound 11) showed potent anti-migratory and anti-invasive activity against MDA-MB-231 at a 100 nM dose in wound-healing and Cultrex BME assay models, respectively. This activity was nearly four times higher than that of compound 1 at the same dose, without cytotoxicity. Latrunculins B and H (compounds 20 and 14) showed unique activity in a G2/M assay module. This assay measures HeLa cell proliferation, apoptosis, DNA content and condensation, cyclin B, and phosphor-histone H3. Both compounds showed potent anti-angiogenic activity, with an IC₅₀ range of 0.04-0.82 μM. Latrunculin B showed potent inhibitory activity for alkaline phosphatase in C2C12 and HuADSC cells, with an EC₅₀, of 0.091 and 0.141 μM, respectively.

We attribute the improved activity of compounds 3 and 14 to their enhanced actin binding affinity. N-p-methoxyphenylacetyl-15-O-methyllatrunculin A (compound 11) had potent anti-invasive activity against both the human metastatic breast cancer cell line MDA-MB-231 and prostate cancer cell line PC-3, without cytotoxicity or effect upon normal cells' viability. The anti-invasive activity of compound 11 was nearly four times greater than that of the parent compound 1 at a 100 nM dose. N-Hydroxymethyllatrunculin A (compound 14, latrunculin H, NSC 749377) showed antiproliferative activity against 18 of 60 cell lines tested at a dose of 10 μM. Five-points dose-response testing of compound 14 showed potent activity against breast, CNS, colon, lung, melanoma, ovarian, and renal cancer cells, with log₁₀GI₅₀ in the range −7.13 to −7.83. Latrunculin H has also been entered in toxicity tests in animal studies. 17-O-Phenylethyllatrunculin A (compound 3, NSC 751494) showed similar activity against the same 18 cell lines, with log₁₀GI₅₀ in the range −7.02 to −7.90. Compounds 3 and 14 were also active in invasion and migration assays, but with cytotoxicity and notable cell shape change. The cytotoxicity and anti-invasive activities of compounds 3 and 14 was correlated with their actin polymerization inhibitory activity, unlike compound 11 which showed better anti-invasive activity with the very low actin polymerization binding affinity of at 100 nM and 1 μM treatments.

Latrunculins B and H (compounds 20 and 14, respectively) were tested in a panel of assays including ApoE secretion, anti-angiogenesis, G2/M, Wnt pathway, insulin secretion, and an array of kinase profiling. The G2/M assays include HeLa cell proliferation, apoptosis, DNA content, and condensation, cyclin B, and phosphor-histone H3. Both compounds showed potent antiangiogenic activity with an IC₅₀ range of 0.04-0.82 μM. Neither compound 20 nor compound 14 showed affinity toward any kinase tested. Latrunculin B showed a unique and potent inhibitory activity against alkaline phosphatase in C2C12 and HuADSC cells, with EC₅₀ of 0.091 and 0.141 μM, respectively. Alkaline phosphatases may perhaps be a target of the anti-invasive latrunculin 11.

### BRIEF DESCRIPTION OF THE DRAWINGS

FIG. 1 (Scheme 1) depicts semisynthetic transformations of latrunculin A (compound 1). Reagents and conditions: (a) R—OH, Et₂O—BF₃, rt, (b) 1—NaH, 0° C., THF, 1 h. 2—R—X, rt, (c) AcOH/H₂O/THF (3:1:1), 60° C. (d) Benzoic anhydride, DMAP, anh CHCl₃, rt, 12 h, (e) 35% aq. CH₂O, EtOH, 24 h, 60° C.

FIGS. 2(A) and 2(B) depicts the time course of actin polymerization in the presence of compounds 1 (FIG. 2(A)) and 3 (FIG. 2(B)), at 0.1 and 1.0 μM doses. Polymerization was monitored by increase in fluorescence intensity (♦: 0 μM, ▪: 0.1 μM, ▴: 1 μM).

FIG. 3 depicts the anti-invasive activities of latrunculin and several derivatives (compounds 1-14) against MDA-MB-231 using the Cultrex™ assay kit. Error bars indicate the SEM for n=3 for each concentration.

### DETAILED DESCRIPTION

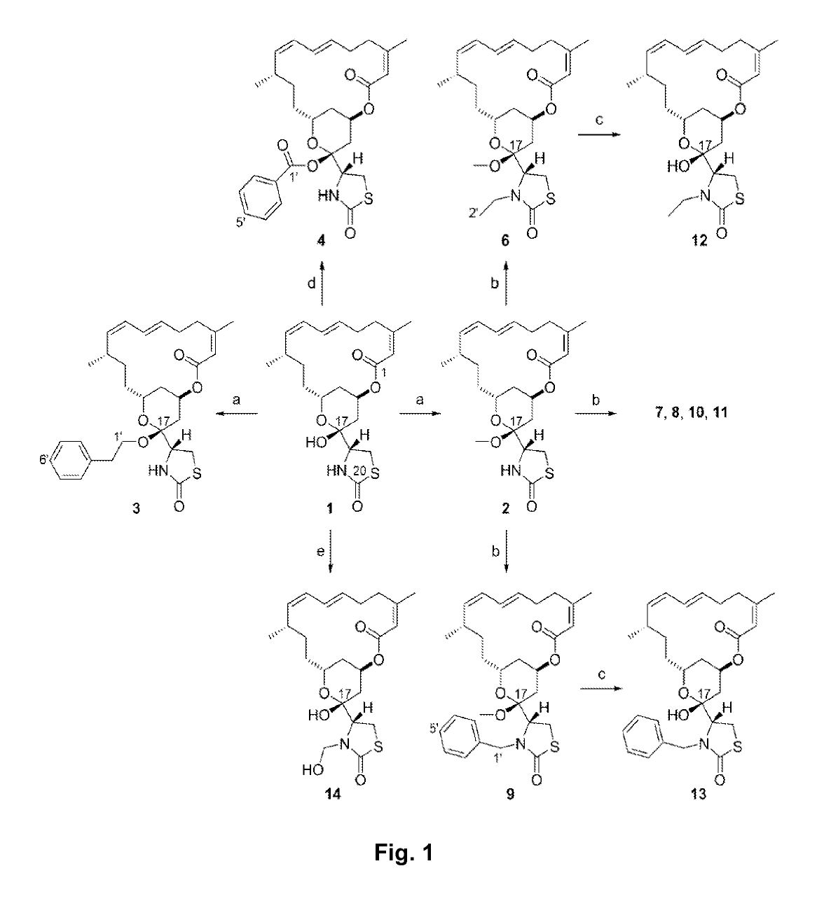

We prepared several semisynthetic derivatives of latrunculin A with diverse steric, electrostatic, hydrogen bond donor (HBD), and hydrogen bond acceptor (HBA) properties. Several C-17 lactol hydroxyl or thiazolidinone NH-substituted latrunculin A derivatives were semisynthesized, compounds 2-14. See FIG. 1 (Scheme 1) and Table 1. Analogs were designed to modulate binding affinity toward G-actin. Examples of the reactions used in the semisynthesis were esterification, acetylation, and N-alkylation. Alternative semisynthetic routes include reaction schemes proceeding via lactol hydroxy and N-substituted derivatives. The semisynthetic latrunculins have been tested for their ability to inhibit pyrene-conjugated actin polymerization, and for their antiproliferative and anti-invasive properties against MCF7, MDA-MB-231, and PC-3 cells using MTT and invasion assays, respectively.

TABLE 1
Structures of Latrunculin A Derivatives.

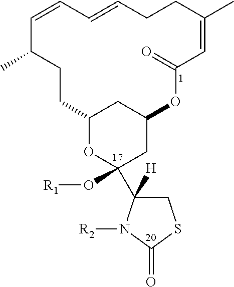

| Comp. | R₁ | R₂ |
|---|---|---|
| 1 | H | H |
| 2 | CH₃ | H |
| 3 | a | H |
| 4 | b | H |
| 5 | CH₃ | CH₃ |
| 6 | CH₃ | CH₂CH₃ |
| 7 | CH₃ | c |
| 8 | CH₃ | (CH₂)₃OH |
| 9 | CH₃ | d |
| 10 | CH₃ | b |
| 11 | CH₃ | e |
| 12 | H | CH₂CH₃ |
| 13 | H | d |
| 14 | H | CH₂OH |
| 15 | CONHCH₂CH₂Cl | H |
| 16 | CONHCH₂CH₂Br | H |
| 17 | CONH(CH₂)₃Cl | H |
| 18 | CONHC₆H₅ | H |
| 19 | CONHCH₂C₆H₅ | H |
| 26 | f | CH₂OH |
| 27 | g | CH₂OH |
| 28 | H | h |
| 29 | a | CH₂OH |
| 30 | (CH₂)₄COOH | H |
| 31 | i | H |
| 32 | H | CH₂NH₂ |
| 33 | H | j |
| 34 | CH₃ | k |

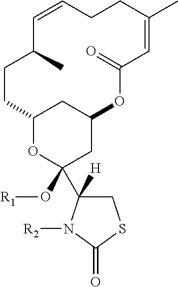

| Comp. | R₁ | R₂ |
|---|---|---|
| 20 | H | H |
| 21 | H | COCH₃ |
| 22 | CH₃ | H |
| 23 | (CH₂)₃CH₃ | H |
| 24 | (CH₂)₇CH₃ | H |
| 25 | H | CH₂OH |

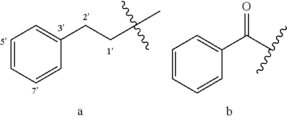

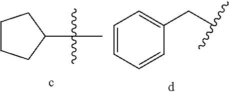

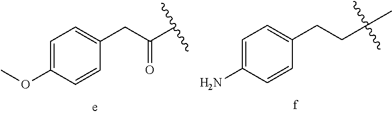

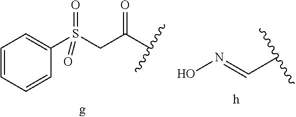

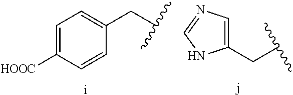

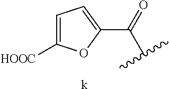

Collection

The Red Sea sponge Negombata magnifica was collected at the Egyptian coast at Hurghada in June to afford a high dry-weight yield of latrunculin A, ˜2 to 3%. At this time of the year there are only low levels of latrunculin B (0.0-0.024% dry weight yield), which saved time, effort, and resources during purification, e.g., through reverse phase chromatography or HPLC chromatographic methods.

Collections from Hurghada, Safaga, and Qusier in December gave high yields of latrunculin B, ˜2% dry weight yield, with only low concentrations of Latrunculin A (0.0027-0.0052%).

Isolation of Latrunculins.

N. magnifica extract is subjected to vacuum liquid chromatography using n-hexane with increasing concentrations of ethyl acetate. Latrunculin-containing fractions are identified by TLC, and subjected to MPLC using isocratic 4% MeOH in CHCl₃. Where necessary, HPLC final purification is conducted semi-preparatively on a Dionex Summit III HPLC unit, with a Phenomenex Luna 250×10 mm, C18 column, and isocratic CH₃CN—H₂O (5:95) elution with UV detection at 235 nm. TLC analysis is carried out using the developing systems n-hexane-EtOAc (1:1) or CHCl₃-MeOH (9:1). For column chromatography, Si gel 60 (particle size 63-200 μm) or Bakerbond octadecyl (C₁₈), 40 μm, is used. Reactions are monitored by TLC. After complete depletion of the starting material, the mixture is diluted with brine and extracted with CHCl₃. The dry extract is fractionated on Si gel 60 using MPLC.

Chemistry

Latrunculin A (compound 1) and latrunculin B (compound 20) were isolated from the Red Sea marine sponge Negombata magnifica, and identified by detailed 1D and 2D nuclear magnetic resonance (NMR) studies and a comparison with literature descriptions. Reaction of compound 1 with methanol or phenethyl alcohol in the presence of BF₃-diethyl etherate (Et₂O—BF₃) yielded the corresponding 17-methoxy analog compound 2 and the phenethoxy derivative compound 3 (Scheme 1, FIG. 1). The configuration at C-17 is maintained after acetalization of the C-17 hydroxyl group, as confirmed by molecular modeling, NMR measurements, and circular dichroism measurements. The distance between H-18 and the C-17 methoxy group in derivative 2 was calculated (using Sybyl) as 2.403 Å, within the range for NOE coupling. The H-18 and C-17 methoxy group showed space dipole-dipole coupling in NOESY measurements. Additionally, the ¹³C chemical shift of C-17 in compound 2 (δ_{C} 99.9) was almost identical to the reported value (δ_{C} 99.8). This observations indicated the preservation of the C-17 configuration between the natural and semi-synthetic derivatives.

High resolution mass spectrometry (HRMS), ¹H NMR data, and ¹³C NMR data for compound 3 indicated the presence of a 17-O-phenethyl side chain. The downfield shift of the C-17 carbon and H-18 signals in compound 3 (+3.2 and 0.20 ppm, respectively), compared with those for the starting material compound 1, suggested possible etherification at C-17. The doublet of triplets of oxygenated methylene H₂-1′ (δ_{H} 3.81, 3.51) showed COSY coupling with the benzylic H₂-2′ triplet (δ_{H} 2.89). Protons H₂-1′ showed ³J HMBC correlations with C-17 and the aromatic quaternary carbon C-3′ (δ_{C} 138.3). Protons H₂-2′ showed ³J HMBC correlation with the aromatic methine carbons C-4′/8′ (δ_{C} 128.9). Protons H-4′/H-8′ showed COSY couplings with protons H-5′/7′ and ³J HMBC correlation with C-6′ (δ_{C} 126.7).

Compound 4 was prepared by treating compound 1 with benzoic anhydride in CHCl₃ in the presence of 4-dimethylaminopyridine (DMAP) as catalyst (Scheme 1). ¹H and ¹³C NMR data indicated benzoylation at C-17. The HRMS data of compound 4 suggested the molecular formula C₂₉H₃₅NO₆S. The aromatic double doublet H-3′/7′ (δ_{H} 7.69) showed COSY coupling with protons H-4′/6′ (δ_{H} 7.42) and ³J HMBC correlation with the carbonyl carbon C-1′ (δ_{C} 169.2). Protons H-4′/6′ in turn showed COSY coupling with H-5′ (δ_{H} 7.53) and ³J HMBC correlation with the aromatic quaternary carbon C-2′ (δ_{C} 133.4). The downfield shift of H-18 (δ_{H} 5.07, >+1.00 ppm) compared with that of the starting material compound 1 was possibly due to the anisotropic effect of the newly introduced C-17-O-benzoyl functionality.

N-Substitution of compound 2 was carried as shown in Scheme 1 using NaH in anhydrous THF at 0° C. and the corresponding alkyl and aryl halides to produce compounds 5-11. The identity of compound 5 was confirmed by NMR data analysis, which showed the replacement of the thiazolidinone NH proton (δ_{H} 5.80) with an N-methyl singlet (δ_{H} 2.97 and δ_{C} 36.7) and comparison with literature. Similarly, the N-ethyl functionality in compound 6 was evident from the COSY coupling between the downfield nitrogenated methylene H₂-1′ (δ_{H} 3.71 and 3.35) and the methyl triplet H₃-2′ (δ_{H} 1.17). Protons H₂-1′ also showed ³J HMBC correlation with the carbonyl C-20, connecting the new ethyl group with the thiazolidinone ring.

The HRMS data of compound 7 suggested an additional degree of unsaturation. Analysis of ¹H and ¹³C data further confirmed the presence of a new N-cyclopentyl moiety. The methine quintet H-1′ (δ_{H} 3.88) showed ³J HMBC correlations with the thiazolidinone carbonyl carbon at δ_{C} 171.5 and the methylene carbons C-3′/4′ (δ_{C} 24.5). Protons H₂-3′/4′ showed COSY coupling with both H₂-2′/5′ protons, which in turn COSY-coupled with H-1′.

¹H and ¹³C data for compound 8 suggested N-hydroxypropylation of the starting material 2. The new methylene singlet H₂-1′ (δ_{H} 3.55) showed a ³J HMBC correlation with C-29 carbonyl (δ_{C} 170.2), connecting this moiety to the thiazolidinone ring. Protons H₂-1′ also showed a ³J HMBC correlation with the oxygenated methylene carbon C-3′ (δ_{C} 59.2). The two chemically nonequivalent H₂-3′ at (δ_{H} 3.76 and 3.58) showed COSY coupling with H₂-2′ (δ_{H}1.78), confirming the introduction of the new N-hydroxypropyl side chain.

High resolution electrospray ionization mass spectrometry (HRESIMS) of compound 9 suggested the molecular formula C₃₀H₃₉NO₅S, twelve degrees of unsaturation, and the N-benzylation of compound 2. The downfield benzylic nitrogenate methylene protons H₂-1′ (δ_{H} 5.11 and 4.35) showed a ³J HMBC correlation with C-20 carbonyl (δ_{C} 170.2), connecting this moiety to the thiazolidinone ring. Protons H₂-1′ also showed a ³J HMBC correlation with the symmetric aromatic methine carbons C-3′/C-7′ (δ_{C} 128.4). Protons H-3′/7′ showed a ³J HMBC correlation with the aromatic methine carbon C-5′ (δ_{C} 127.7), and COSY correlation with protons H-4′/6′ (δ_{H} 7.34). The latter protons also showed COSY correlation with H-5′ (δ_{H} 7.31) and a ³J HMBC correlation with the quaternary aromatic carbon C-2′ (δ_{C} 132.5).

¹H and ¹³C NMR data for compound 10 were similar to those for compound 9, with an N-benzoyl functionality replacing the N-benzyl of compound 9. The aromatic methine protons H-3′/7′ (δ_{H} 7.72) showed a ³J HMBC correlation with the carbonyl carbon C-1′ (δ_{C} 169.7). The benzoylation of the C-17 lactol hydroxy in compound 4 and of the thiazolidinone NH in compound 10 resulted in a significant downfield shifting of proton H-18 (+1.22 and +1.49 ppm, respectively) compared with that of compound 1, possibly due to the anisotropic effect of the benzene ring and the carbonyl group.

Reaction of compound 2 with p-methoxy phenylacetyl chloride produced compound 11, which was highly unstable in the reaction pot, with the amide bond rapidly degrading. We therefore shortened the reaction time to five minutes after reagent was added. The HRESIMS data of compound 11 suggested the molecular formula C₃₂H₄₁NO₇S. ¹H and ¹³C NMR data of compound 11 suggested there had been a successful N-p-methoxy phenylacetylation. The methoxy singlet H₃-9′ (δ_{H} 3.77) showed a ³J HMBC correlation with quaternary aromatic oxygenated carbon C-6′ (δ_{C} 159.8). The aromatic protons H-4′/8′ (δ_{H} 7.21) showed ³J HMBC correlations with C-6′ and the benzylic methylene C-2′ (δ_{C} 42.3). They also show COSY coupling with protons H-5′/7′ (δ_{H} 6.81). Proton singlet H₂-2′ (δ_{H} 3.79) showed ²J HMBC correlations with the carbonyl carbon C-1′ (δ_{C} 175.7) and the quaternary aromatic carbon C-3′ (δ_{C} 126.3). Proton H-18 (δ_{H} 5.30) showed a ³J HMBC correlation with C-1′ carbonyl, connecting the new p-methoxy phenylacetyl moiety with the thiazolidinone ring.

To explore the effect of N-substitution on the pharmacological activity of the unsubstituted C-17 lactol group, compounds 6 and 9 were demethylated by heating with aqueous AcOH to produce compounds 12 and 13, respectively, with a free C-17 lactol hydroxyl functionality (Scheme 1). Compounds 12 and 13 showed ¹H and ¹³C NMR spectra essentially identical to those of compounds 6 and 9, respectively, but with the C-17 methoxy replaced with a hydroxyl group. The D₂O-exchangeable broad proton singlets at δ 4.01 and 3.91 were assigned as the new C-17 hydroxy signals in compounds 12 and 13, respectively.

N-Hydroxymethyl latrunculin A (compound 14) was prepared as described in D. Blasberger et al., Liebigs Ann. Chem. 1989, 12, 1171-1188, to study the effect of extending the location of the only HBD in compound 1 (the thiazolidinone NH) by one carbon, and replacing it with a primary alcohol group (See Scheme 1).

Biological Activity

Compound 1 reversibly binds cytoskeleton actin monomers, forming a 1:1 complex with G-actin, and disrupting its polymerization. Compound 1 has shown antiangiogenic, antiproliferative, antimicrobial, and anti-migratory activities. Compound 1 and the other latrunculins and derivatives described here will inhibit cancer and other diseases whose pathology is linked to the stability of the cytoskeleton actin.

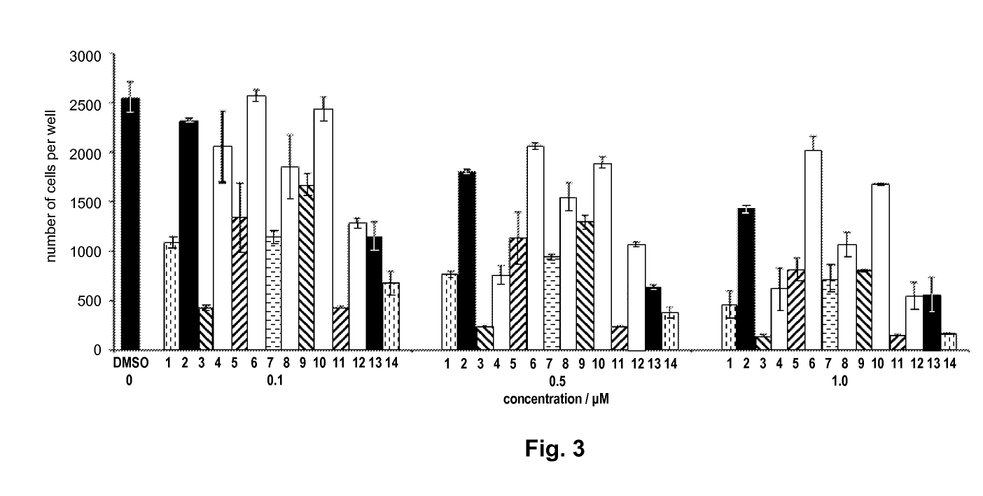

An actin polymerization kit was used to assess the direct actin-binding activity of each analogue at two concentrations: 100 nM and 1 μM. This kit is based on the enhanced fluorescence of pyrene-conjugated actin that occurs during polymerization. When pyrene-G-actin (monomer) forms pyrene-F-actin, fluorescence is enhanced. The enhanced fluorescence is used to follow polymerization over time. Table 2 lists activities as the percentage of actin polymerization inhibition, compared to the negative vehicle control (DMSO), along with calculated IC₅₀. FIG. 3 shows the time-course inhibition of actin polymerization by compounds 1 and 3. Compounds 3 and 14 were substantially more active than the parent latrunculin A (compound 1) in inhibiting actin polymerization at both doses, while compounds 4, 9, 12, and 13 were almost equipotent to 1. This specific binding assay demonstrates that the antiproliferative and anti-invasive activities of latrunculin A and its derivatives are generally due to the disruption of actin polymerization, with some exceptions, such as compound 11.

TABLE 2
Pyrene Actin Polymerization Assay of Latrunculin A Derivatives.

| | Actin polymerization inhibition (%) | | |
|---|---|---|---|
| compound | 100 nM | 1 μM | IC₅₀ (nM)^{[a]} |
| 1 | 30 | 73 | 284 ± 72 |
| 2 | 6 | 21 | 9233 ± 825 |
| 3 | 31 | 94 | 184 ± 48 |
| 4 | 23 | 67 | 466 ± 51 |
| 5 | 11 | 19 | >10000 |
| 6 | 0 | 18 | >10000 |
| 7 | 20 | 49 | 1049 ± 271 |
| 8 | 0 | 21 | 8548 ± 574 |
| 9 | 33 | 67 | 333 ± 76 |
| 10 | 17 | 53 | 834 ± 109 |
| 11 | 0 | 13 | >10000 |
| 12 | 32 | 70 | 283 ± 51 |
| 13 | 24 | 64 | 445 ± 79 |
| 14 | 35 | 90 | 210 ± 54 |

^{[a]}IC₅₀ values, in nM, were determined from a nonlinear dose-response curve using GraphPad Prism. Values reported are mean ± SEM. Each experiment was conducted in triplicate.

The antiproliferative and anti-invasive activities of compounds 1-14 were quantified using MTT and cell invasion assay kits, respectively. Latrunculin A (compound 1) was used as a positive control in all biological assays, because it is known to be an actin polymerization inhibitor, with cytotoxic, antiproliferative, and anti-invasive activities. The 3-[4,5-dimethylthiazol-2-yl]-2,5-diphenyl tetrazolium bromide (MTT) assay selectively detects living cells in a quantitative, colorimetric fashion, based on a living cell's ability to reduce MTT to an insoluble, purple formazan dye. The MTT assay is routinely used to assess cytotoxicity and proliferation inhibition activities. The antiproliferative activities of compounds 1-14 were measured against two human mammary gland adenocarcinoma cell lines, MCF7 and MDA-MB-231, and against human prostate cancer cells PC-3 at five different doses (0.1, 0.5, 1, 10, and 50 μM). The IC₅₀ values are shown in Table 3. While many of the semisynthetic latrunculins had similar activity levels, some had greater activity than compound 1. In particular, compounds 3 and 14 were both more potent than compound 1 against both cell lines, with 4.8 and 4.0-fold increases in the activity against MCF7, respectively, and with noticeable cytotoxic effect at doses higher than 1 μM. Although the MDA-MB-231 cells were more resistant than MCF7 against most latrunculin derivatives tested, compound 14 had potent activity against both cell lines. Compounds 3 and 14 were both more potent than compound 1 against prostate cancer cells PC-3, with 3.2 and 10.8-fold increases in activity, respectively. Although derivative 10 was less active than compound 1 in the invasion assay, compounds 10, 4, and 9 were almost equipotent with the parent natural product 1 in the proliferation assay.

TABLE 3
Antiproliferative Activities of Latrunculin A Derivatives.

| | IC₅₀ (μM) ^{[a]} | | |
|---|---|---|---|
| compound | MCF7 | PC-3 | MDA-MB-231 |
| 1 | 0.48 ± 0.04 | 3.90 | 4.19 ± 1.49 |
| 2 | 10.19 ± 1.72 | 22.3 | 23.42 ± 0.85 |
| 3 | 0.10 ± 0.07 | 1.22 | 2.71 ± 0.52 |
| 4 | 0.52 ± 0.14 | 6.44 | 5.05 ± 0.36 |
| 5 | 13.07 ± 3.69 | 19.41 | 19.93 ± 3.27 |
| 6 | 20.64 ± 2.77 | 29.3 | >50.0 |
| 7 | 5.30 ± 1.32 | 7.24 | 5.07 ± 0.72 |
| 8 | 19.20 ± 3.91 | 34.2 | >50.0 |
| 9 | 0.78 ± 0.24 | 8.30 | 6.82 ± 1.41 |
| 10 | 0.55 ± 0.091 | 4.70 | 4.48 ± 1.79 |
| 11 | 15.4 ± 1.21 | 19.8 | 26.17 ± 3.91 |
| 12 | 0.95 ± 0.28 | 5.72 | 7.19 ± 1.01 |
| 13 | 0.63 ± 0.02 | 3.8 | 2.72 ± 0.39 |
| 14 | 0.12 ± 0.01 | 0.36 | 2.99 ± 0.15 |

^{[a]} IC₅₀ values, in μM, were determined from a nonlinear dose-response curve using GraphPad Prism. Values reported are mean ± SEM. Each experiment was conducted in triplicate.

The anti-invasive activities of compounds 1-14 were measured using a 96 well basement membrane extract (BME) cell invasion assay kit against the highly metastatic MDA-MB-231 cells. This assay employs a simplified Boyden Chamber with a polyethylene terephthalate membrane (8 μm pore size). Cell invasion was quantified using calcein-acetomethylester (calcein-AM). Cells internalize calcein-AM; intracellular esterases then cleave the acetomethylester moiety to generate free calcein. Free calcein fluoresces brightly. This fluorescence may be used to quantify the number of cells that have invaded across the BME. The anti-invasive activities of compounds 1-14 at three different doses are shown in FIG. 3. To correlate the anti-invasive activities of the latrunculin A derivatives with their actin-disrupting activities, compounds 1-14 were each tested at non-toxic concentrations. Except for compound 3, the MTT assay showed no or insignificant toxicity at concentrations below 1.0 μM over a period of 72 hours. Accordingly, all compounds were tested at 0.1, 0.5, and 1.0 μM. Compound 3 killed 17% of MDA-MB-231 cells at 1.0 μM, while the same dose of compound 3 inhibited 95% of MDA-MB-231 cells' invasion. These observations showed a clear difference between the cytotoxic and anti-invasive activity levels. The anti-invasive activities of compounds 1-14 are attributed primarily to the inhibition of actin polymerization, not to cell death per se.

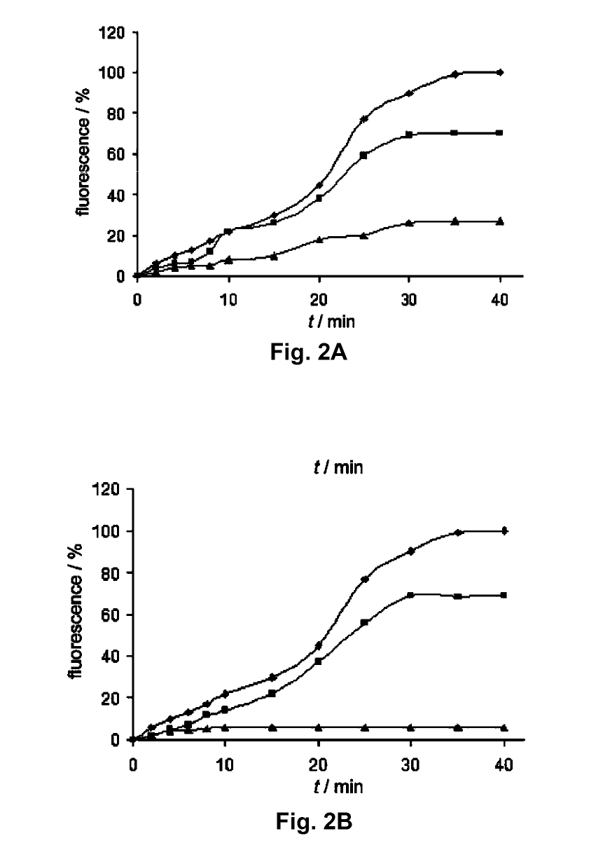

Derivatives 3, 11, 13, and 14 were 3.3, 4.6, 3.1, and 2.1-fold more active, respectively, against cell invasion than latrunculin A (compound 1) at 0.5 μM. 17-O-Phenylethyl-latrunculin A (compound 3) was the most potent, with a 100 nM dose inhibiting MDA-MB-231 cell invasion, as compared to a 1 μM dose of compound 1. Upon the addition of compound 1, 3, or 14 to MDA-MB-231 cells in the upper assay chamber, rapid changes in cell morphology were seen; actin filaments were disrupted, as characterized by remarkable cell deformity (data not shown). During cell migration, cytoskeletal actin dynamically reorganizes to impart the forces that cause cell migration. These observations suggest that the anti-invasive activities of compounds 1, 3, and 14 are mediated through direct disruption of actin cytoskeleton remodeling. While compounds 4 and 12 were equipotent to compound 1 at 1 and 0.5 μM doses, the others were less active. Interestingly, the second most active derivative, N-p-methoxyphenylacetyl-15-O-methyl-latrunculin A (compound 11) showed potent anti-invasive activity at a 100 nM dose (FIG. 2). This activity was nearly 4-fold greater than that of compound 1 at the same dose, but without antiproliferative, cytotoxic, or cell shape modifying activities. Although compound 11 was dramatically less active than compound 1 in the antiproliferation assay, it was nevertheless surprisingly potent in attenuating the metastasis of MDA-MB-231 cells. Because compound 11 inhibits actin polymerization only weakly (See Table 2), compound 11 presumably has different target(s), target(s) other than the microfilament actin.

N-Substitution of compound 1 with ethyl and benzyl groups preserves the potency, with IC₅₀ values of 0.63 μM and 0.95 μM for compounds 13 and 12, respectively, against MCF7. MDA-MB-231 was more resistant, with IC₅₀ values of 2.72 μM for compound 13, and 7.19 μM for compound 12.

Latrunculin derivative 14 showed greater activity than the parent latrunculin A (compound 1) by factors of 4 and 1.4, respectively, against MCF7 and MDA-MB-231 cells in the proliferation assay; and by a factor of 2.7 against MDA-MB-231 cells in the invasion assay; all assessed at 1 μM. The pronounced activity of compound 14 may be due to improved direct actin binding via the more approachable HBD N-hydroxymethylene as compared to the thiazolidinone NH in compound 1.

Experimental Procedures.

Optical rotation was measured on a Rudolph Research Analytical Autopol III polarimeter. IR spectra were recorded on a Varian 800 FT-IR spectrophotometer. ¹H and ¹³C NMR spectra were recorded in CDCl₃, using TMS as an internal standard, on a JEOL Eclipse NMR spectrometer operating at 400 MHz for ¹H, and 100 MHz for ¹³C. The HREIMS experiments were conducted on a Micromass LCT spectrometer. Analytical HPLC analyses were performed on a DIONEX® Summit II system using a Phenomenex Luna 250×4.6 mm, RP-C-18 column, isocratic elution (100% MeOH), and UV detection set at 235 nm to verify the purity of each Latrunculin compound. Each of latrunculins 1-14 had a purity of >98%, except for analogue 9, which had 91% purity. TLC analysis was carried out on precoated Si gel 60 F₂₅₄ 500 μm TLC plates (EMD Chemicals), using the developing systems n-hexane-EtOAc (1:1) or CHCl₃-MeOH (9:1). For column chromatography, Si gel 60 (EMD Chemicals, 63-200 μm), fine Si gel 60 (EM Science, <63 μm), and C-18 Si gel (Bakerbond, Octadecyl 40 μm) were used. For Sephadex LH-20 column chromatography, n-hexane-CHCl₃ (1:3), CHCl₃, and CHCl₃-MeOH (9:1) solvent systems were used.

General Chemical Reaction Procedures. (A) Acetalization of the C-17 hydroxyl group of latrunculin A: To a solution of compound 1 in ROH, BF₃-diethyl etherate (Et₂O—BF₃) was added. The mixture was stirred for 12 h at room temperature, and was neutralized with 10% aq. NaHCO₃ solution. The solvent was evaporated, and the residue was extracted with CHCl₃ and dried over MgSO₄. After solvent evaporation the residue was chromatographed on a Si gel 60 column.

(B) Alkylation of the thiazolidinone nitrogen: A solution of compound 2 in dry THF was gradually added to a suspension of NaH (60% dispersion in mineral oil) in dry THF at 0° C. The mixture was then stirred for 1 h at 0° C. Alkyl halide was added and the mixture stirred at room temperature until compound 2 was completely depleted. Ether and water were then added, the two layers were separated, and the organic layer was dried over Na₂SO₄. Evaporation of the solvents left a residue, which was chromatographed on Si gel 60 column.

(C) Demethylation of 17-O-methyllatrunculin A derivatives: A solution of 17-O-methyllatrunculin A analogue in AcOH/H₂O/THF (3:1:1) was stirred and heated to 60° C. The reaction was monitored by TLC until complete depletion of the starting material (˜0.5-1 h). The mixture was then cooled to room temperature, neutralized with 10% aqueous NaHCO₃ solution, and ether was then added. The upper organic layer was then washed with water, dried over anhydrous Na₂SO₄, and evaporated under reduced pressure. The residue was chromatographed on a Si gel 60 column.

17-O-Methyllatrunculin A (compound 2): Compound 2 was prepared according to procedure A from compound 1 (200 mg, 0.72 mmol), MeOH (5 mL), and Et₂O—BF₃ (0.22 mL, 1.77 mmol). Elution with n-hexane/EtOAc (8:2) afforded compound 2 (144 mg, 72.0%).

17-O-Phenylethyllatrunculin A (compound 3): Compound 3 was synthesized according to the general procedure A from 1 (15 mg, 0.036 mmol), phenethanol (1 mL, 7.131 mmol), and Et₂O—BF₃ (15 μL, 0.117 mmol). Elution with n-hexane/EtOAc (8:2) afforded 3 (5.5 mg, 36.7%): colorless oil, [α]_{D}²⁵ +34.5 (c=0.18 in CHCl₃). NMR, IR, and MS data are presented in the 61/267,575 priority application.

17-O-Benzoyllatrunculin A (compound 4): A solution of compound 1 (20 mg, 0.047 mmol) in anhydrous CHCl₃ (3 mL) was stirred with benzoic anhydride (11 mg, 0.050 mmol) and a catalytic amount of DMAP for 12 hours at room temperature. The reaction mixture was neutralized with NaHCO₃ solution. The organic layer was washed with H₂O (2×5 mL), dried over anhydrous Na₂SO₄, and evaporated under reduced pressure. The reaction residue was chromatographed over Si gel 60 using isocratic n-hexane/EtOAc (9:1) to afford compound 4 (4.7 mg, 23.5%): colorless oil, [α]_{D}²⁵ +47.1 (c=0.25 in CHCl₃). NMR, IR, and MS data are presented in the 61/267,575 priority application.

17-O-Methyl-N-methyllatrunculin A (compound 5): Compound 5 was prepared according to procedure B using compound 2 as starting material (10 mg, 0.022 mmol), in THF (1 mL), NaH (2 mg, 0.041 mmol) in THF (1 mL) and MeI (0.1 mL, 1.600 mmol). Elution with n-hexane/EtOAc (8:2) afforded compound 5 (7.1 mg, 70.1%).

17-O-Methyl-N-ethyllatrunculin A (compound 6): Compound 6 was prepared according to procedure B using compound 2 as starting material (10 mg, 0.022 mmol) in THF (1 mL), NaH (2 mg, 0.041 mmol) in THF (1 mL), and Etl (0.1 mL, 1.147 mmol). Elution with n-hexane/EtOAc (8:2) afforded compound 6 (7.6 mg, 70.6%): colorless oil, [α]_{D}²⁵ +33.7; (c=1.3 in CHCl₃). NMR, IR, and MS data are presented in the 61/267,575 priority application.

17-O-Methyl-N-cyclopentanelatrunculin A (compound 7): Compound 7 was prepared according to procedure B starting with compound 2 (10 mg, 0.022 mmol), in THF (1 mL), NaH (2 mg, 0.041 mmol) in THF (1 mL) and iodocyclopentane (0.1 mL, 0.867 mmol), elution with n-hexane/EtOAc (8:2) afforded compound 7 (7.6 mg, 70.6%): colorless oil, [α]_{D}²⁵ +53.6 (c=0.63 in CHCl₃). NMR, IR, and MS data are presented in the 61/267,575 priority application.

17-O-Methyl-N-(3′-hydroxypropyl)latrunculin A (compound 8): Compound 8 was prepared according to procedure B from compound 2 (10 mg, 0.022 mmol) in THF (1 mL), NaH (2 mg, 0.041 mmol) in THF (1 mL), and 3-iodo-1-propanol (0.1 mL, 1.043 mmol). Elution with n-hexane/EtOAc (8:2) afforded compound 8 (4.6 mg, 40.6%): colorless oil, [α]_{D}²⁵ +53.6 (c=0.83 in CHCl₃). NMR, IR, and MS data are presented in the 61/267,575 priority application.

17-O-Methyl-N-benzyllatrunculin A (compound 9): Compound 9 was prepared according to procedure B using compound 2 as starting substrate (15 mg, 0.033 mmol) in THF (1 mL), NaH (2 mg, 0.041 mmol) in THF (1 mL), and benzyl chloride (0.1 mL, 0.866 mmol). Elution with n-hexane/EtOAc (9:1) afforded compound 9 (11.3 mg, 75.3%): colorless oil, [α]_{D}²⁵ +28.5 (c=0.48 in CHCl₃). NMR, IR, and MS data are presented in the 61/267,575 priority application.

17-O-Methyl-N-benzoyllatrunculin A (compound 10): Compound 10 was prepared according to procedure B using compound 2 as starting substrate (10 mg, 0.022 mmol) in THF (1 mL), NaH (2 mg, 0.041 mmol) in THF (1 mL), and benzoyl chloride (0.1 mL, 0.866 mmol). Elution with n-hexane/EtOAc (8:2) afforded compound 10 (5.2 mg, 52.0%): colorless oil, [α]_{D}²⁵ +42.7 (c=0.15 in CHCl₃). NMR, IR, and MS data are presented in the 61/267,575 priority application.

17-O-Methyl-N-(7′-methoxyphenylacetyl)latrunculin A (compound 11): Compound 11 was prepared according to procedure B from compound 2 (10 mg, 0.022 mmol) in THF (1 mL), NaH (2 mg, 0.041 mmol) in THF (1 mL), and p-methoxyphenylacetyl chloride (0.1 mL, 0.636 mmol). Elution with n-hexane/EtOAc (8:2) afforded compound 11 (2.2 mg, 22.0%): Colorless oil, [α]_{D}²⁵ +63.6 (c=0.1 in CHCl₃). NMR, IR, and MS data are presented in the 61/267,575 priority application.

N-Ethyllatrunculin A (compound 12): Compound 12 was prepared according to procedure C starting with compound 6 (5 mg, 0.011 mmol) in acidic solution (1 mL) for 3 h at 60° C. Elution with n-hexane/EtOAc (9:1) afforded compound 12 (1.3 mg, 26.0%): colorless oil, [α]_{D}²⁵ +63.6 (c=0.11 in CHCl₃). NMR, IR, and MS data are presented in the 61/267,575 priority application.

N-Benzyllatrunculin A (compound 13): Compound 13 was prepared according to procedure C from compound 9 (9 mg, 0.0176 mmol) in acidic solution (1 mL) for 5 h at 60° C. Elution with n-hexane/EtOAc (9:1) afforded compound 13 (1.8 mg, 20.0%): colorless oil, [α]_{D}²⁵ +63.6 (c=0.17 in CHCl₃). NMR, IR, and MS data are presented in the 61/267,575 priority application.

N-Hydroxymethyllatrunculin A (compound 14): A solution of 10 mg of compound 1 in 3 mL EtOH was treated with 3 mL 35% aq. CH₂O, and stirred for 24 hours at 60° C. Brine (5 mL) was then added, and the mixture was extracted with CHCl₃ (2×5 mL). The residue was chromatographed over Si gel 60 with an n-hexane/EtOAc (8:2) solvent system to afford compound 14 (2.8 mg, 28%): colorless oil, [α]_{D}²⁵ +70 (c=2.2 in CHCl₃). NMR, IR, and MS data are presented in the 61/267,575 priority application.

Pyrene-Actin Polymerization Assays. Actin polymerization assays were performed in accordance with the manufacturer's instructions (Cytoskeleton, Denver, Colo.). Briefly, 5 mM final concentration of monomeric actin (1:10 pyrene labeled) was incubated on ice for 10 min with one of various concentrations of one of the latrunculin derivatives. Samples were then equilibrated 10 min in an ELISA plate reader (BioTek, VT), after which polymerization was induced by adding KCl, MgCl₂, and ATP. Compounds 1-14 were tested at 0.1, 0.5, 1.0, and 10 μM. The IC₅₀ for each compound was calculated using GraphPad Prism 5.0.

Cell Cultures. Breast cancer cell lines MCF7 and MDA-MB-231 were purchased from ATCC (Manassas, Va.). The cell lines were grown at 37° C. under 5% CO₂ in 10% fetal bovine serum (FBS) and RPMI 1640 (GIBCO-Invitrogen, NY) supplemented with 2 mmol/L glutamine, 100 μg/mL penicillin G, and 100 μg/mL streptomycin.

Preparation of Latrunculin a Derivatives for Cell Culture Assays: A Latrunculin derivative was first dissolved in DMSO at 50 mM (for MTT assays) and 1 mM (for Cultrex assays). About 1 μL of the solution was transferred to 1 mL of serum-free medium to obtain 50 μM and 1 μM dilutions (0.001% DMSO) for MTT and Cultrex assays, respectively. Serial dilutions were then conducted to produce various concentrations.

MTT Proliferation Assay. The growth of MCF7, MDA-MB-231, and PC-3 cancer cell lines was measured using an MTT kit (TACS™, TREVIGEN®, Inc.). Exponentially growing cells were plated in a 96-well plate at a density of 8×10³ cells per well, and allowed to attach for 24 h. Complete growth medium was then replaced with 100 μL of RPMI serum-free medium containing various concentrations (50, 10, 1, 0.5, 0.1 μM) of the particular test compound. Culture continued at 37° C. under 5% CO₂. After 72 h of culture, the cells were treated with MTT solution (10 μL/well) at 37° C. for 4 h. The color reaction was stopped by adding solubilization/stop solution (100 μL/well). Incubation continued at 37° C. continued to completely dissolve the formazan dye product. Absorbance of the samples was determined at 570 nm with an ELISA plate reader (BioTek, VT). The number of cells per well was calculated against a standard curve that had been prepared by plating various concentrations of cells, as determined by hemocytometer, at the start of each experiment. IC₅₀ for each compound was calculated using a nonlinear regression curve fit of log concentration versus the number of cells/well, using GraphPad Prism 5.0 (GraphPad Software, La Jolla, Calif.).

Cultrex® Cell Invasion Assay. Anti-invasive activities were measured using Trevigen's Cultrex® Cell Invasion Assay as described in K. Tamilarasan et al., BMC Cell Biol. 2006, 7, 17-30; and P. Borghesani et al., Development. 2002, 6, 1435-1442. About 50 μL of basement membrane extract (BME) coat was added to each well. After incubation for 4 h at 37° C. under 5% CO₂, 50,000 MDA-MB-231 cells in 50 μL of serum free RPMI medium were added to each well in the top chamber, along with the test compound at one of several concentrations (0.1, 0.5, 1 μM). About 150 μL of RPMI medium was added to the lower chamber in 10% FBS and penicillin/streptomycin, using fibronectin (1 μL/mL) and N-formyl-met-leu-phe (10 nM) as chemoattractants. Cells were allowed to migrate to the lower chamber at 37° C. under 5% CO₂. After 24 h, the top and bottom chambers were aspirated and washed with the washing buffer that had been supplied with the kit. About 100 μL of Cell Dissociation Solution/Calcein-AM solution was added to the bottom chamber, and the cells were incubated at 37° C. under CO₂ for 1 h. Briefly, the cells internalize calcein-AM, and then the intracellular esterases cleave the AM moiety to generate free calcein. Fluorescence of the samples was observed at 485 nm excitation, 520 nm emission, using an ELISA plate reader (BioTek, VT). The number of cells that had invaded through the BME coat was calculated using a standard curve.

Molecular Modeling. Three-dimensional structure building and modeling were performed using the SYBYL program package, version 8.0. The X-ray crystallographic structural of compound 1 has been reported: W. M. Morton et al., Nat. Cell Biol. 2000, 2, 376-378. This reported structure was used as a template to model other latrunculins. Conformations for each compound were generated using Confort™ conformational analysis. Energy minimizations were performed using the Tripos force field with a distance-dependent dielectric and the Powell conjugate gradient algorithm, with a convergence criterion of 0.01 kcal/(mol-Å). Partial atomic charges were calculated using the semiempirical program MOPAC 6.0 and applying the AM1. See J. J. Stewart, J. Comput. Aided Mol. Des. 1990, 4, 1-105. Results are presented in the 61/267,575 priority application.

Molecular Docking. The Surflex-Dock program, version 2.0, interfaced with SYBYL 8.0 was used to model the docking of the compounds to the active site of actin. Surflex-Dock employs an idealized active site ligand (protomol) as a target to generate putative poses of molecules or molecular fragments. These putative poses were scored using the Hammerhead scoring function. The program was used to dock the training set molecules into the active site of G-actin. The 3D structure was taken from the Brookhaven protein databank (PDB code: 1 esv).

Semisynthesis of Additional Latrunculin Derivatives.

C-17/C-15 lactol acetalization products are synthesized via acetalization of the lactol group of compound 1 or 20 in a substituted or free alcohol (Ar—(CH₂)₁₋₃—OH or HetAr—(CH₂)₁₋₃—OH), BF₃-diethyl etherate (Et₂O—BF₃). The mixture is stirred for 12 h at room temperature and then neutralized with 10% aq. NaHCO₃. The solvent is evaporated, and the residue is extracted with CHCl₃ and dried over MgSO₄. After evaporation of the solvent, the residue is subjected to MPLC.

Hydroxymethylation/hydroxyethylation is conducted through alkylation of the thiazolidinone nitrogen. A solution of compound 1 or 20 in 35% aqueous formaldehyde or acetaldehyde in ethanol is kept overnight at 60° C. Reaction workup and chromatography will afford 30-40% yield.

Compound 26 is prepared by the acetalization reaction as otherwise described above, using 2-(4-aminophenyl)ethanol after protecting the amino group with Boc, followed by later de-protection. The product is hydroxymethylated with 3% aqueous HCHO.

Compound 27 is prepared by carbamoylation of compound 1 using benzenesulfonylmethyl isocyanate in toluene, followed by hydroxymethylation.

Compound 28 is prepared via oxidation of the N-hydroxymethyl group of compound 14 to an aldehyde using pyridinium chlorochromate in CH₂Cl₂, followed by reaction with NH₂OH.HCl, pyridine-EtOH (1:3), and heating at 100° C. for 18 h.

Compound 29 is prepared by treating compound 3 with 35% aqueous formaldehyde in ethanol for 12-24 hours in a 60° C. water bath. Product is purified on Sephadex LH20 (1-5% MeOH/CHCl₃) or C-18 RP (CH₃CN—H₂O gradient elution). Si gel 60 is not used, to avoid chemical instability or degradation.

Compounds 30 and 31 are prepared by essentially the same procedure used to prepare compound 3, but replacing phenylethyl alcohol with ethyl 5-hydroxypentanoate or ethyl-4-(hydroxymethyl)benzoate, respectively. The ethyl ester is hydrolyzed with acetic acid, HCl (because latrunculins are generally more stable in acids than in bases), aqueous t-BuOK in THF, Me₃SiI, KOSiMe₃, cyclodextrins, or Dowex-50.

Compound 32 is prepared by reacting compound 1 with methanol and Et₂O—BF₃ overnight to give compound 2 in 70-80% yield. Bromination of CH₃NH₂.HCl in ethanol under nitrogen for 5 hours at 40° C. gives bromomethaneamine.HCl in 90% yield. Compound 2 in dry THF is gradually treated with a suspension of NaH (60% dispersion in mineral oil) in dry THF at 0° C. The mixture is then stirred for 1 hour at 0° C. BrCH₂NH₂ is then added, and the mixture is stirred at room temperature until compound 2 is completely depleted. Ether and water are added, the two layers are separated, and the organic layer is dried over anhydrous Na₂SO4. After evaporation the residue is subjected to MPLC on Sephadex LH20 (1-5% MeOH/CHCl₃) or C-18 RP (CH₃CN—H₂O gradient elution) to give the 17-O-methyl analogue of compound 32.

Compound 32. Alternatively, compound 2 is added to bromomethaneamine in anhydrous Et₂O, and the mixture is treated with metallic Na with ice cooling under nitrogen for 3 hours, after which NaOEt is added to give the 17-O-methyl analogue of compound 32, which is then demethylated using AcOH/H₂O/THF (3:1:1) and stirring at 60° C. The reaction is monitored using TLC until complete depletion of the starting material (˜0.5-1 hours). The mixture is cooled to room temperature, neutralized with 10% aqueous NaHCO₃, and extracted with ether. The ether layer is washed with water, dried over anhydrous Na₂SO₄, and evaporated under reduced pressure. The residue is subjected to Sephadex LH20 or C18 reverse phase chromatography to give compound 32.

Compound 33. Compound 2 in dry THF is gradually treated with a suspension of NaH (60% dispersion in mineral oil) in dry THF at 0° C. The mixture is then stirred for 1 hour at 0° C. Commercially available 4-(bromomethyl)imidazole (CAS# 80733-10-44) is then added, and the mixture is stirred at room temperature until compound 2 is completely depleted. Ether and water are added, the two layers are separated, and the organic layer is dried over anhydrous Na₂SO4. After evaporation the residue is subjected to MPLC on Sephadex LH20 (1-5% MeOH/CHCl₃) or C-18 RP (CH₃CN—H₂O gradient elution) to afford the 17-O-methyl analogue of compound 33, which is then demethylated by using AcOH/H₂O/THF (3:1:1) as otherwise described above for compound 32, to afford compound 33. An alternative scheme for the semisynthesis of compound 33 proceeds by the 5% CuI-catalyzed N-methyl-imidazolation of compound 1 directly, using 4-(bromomethyl)-imidazole in the presence of (S)-pyrrolidinylmethylimidazole and Cs₂CO₃ in DMF at 110° C. for 24 hours. This method allows the incorporation of diverse functional groups such as ester, nitrile, nitro, ketone, free OH, and free NH₂ on the halide.

Compound 34: 17-O-Methyl-N-(2′-carboxyfuroate)latruncul in A. Compound 34 is prepared according to procedure B from compound 2, otherwise similar to the method for preparing compound 11, using NaH in THF and 2′-carboxyfuroylchloride and reacting at room temperature for 5 minutes.

Small Molecule Analogs. The invention may also be practiced with small molecule latrunculin analogs. Examples of smaller analogs that may be used in practicing the present invention include the following:

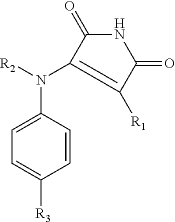

R₁═H, CH₂OH, CHO, COOH, CONH₂, NH₂, C1 to C6 hydroxyalkyl or carboxyalkyl, Hydroxylaryl, Hydroxyheteroaryl,
R₂═H, CH₂OH, CHO, C1 to C6 hydroxyalkyl or carboxyalkyl, Hydroxylaryl, Hydroxyheteroaryl,
R₃═COOH, CHO, CONH₂, OH, O—C1 to C4 Alkyl or hydroxyalkyl, NH₂, NH—C1 to C4 carboxyalkyl or hydroxyalkyl, SH
Our molecular modeling studies suggest that these maleidimide structures will exhibit high binding affinity toward the actin-binding proteins gelsolin, cofilin, and CapG, and other gelosolin-relating actin binding proteins. These compounds are expected to be bioisosteres of the latrunculin active tetrahydropyran/thiazolidinone segment. Therefore they should show selective anti-invasive activity similar to that of compound 11. These analogs may be totally synthesized through methods otherwise known in the art, and therefore should not require harvesting marine fauna to obtain semi-synthetic precursors.

In Vitro Evaluation

The in vitro antiproliferative and anti-invasive activities of various latrunculin analogues are observed and measured. Some preliminary in vitro studies have been conducted, with encouraging results.

Effects on HeLa cells. In an in vitro assay, latrunculin H (compound 14) strongly inhibited the growth of cervical cancer HeLa cells at early interphase over a dose range 0.04-1.25 μM, as analyzed by flow cytometry. Latrunculin 11 showed 3.5-51.0 times greater inhibition against G2-M-phase HeLa cells as compared to the control antimitotic compound nocodazole in various G2_M assays. Latrunculin B (compound 20) and latrunculin H (compound 14) were less active than compound 11 in these assays, but still showed greater activity than did nocodazole.

Effects on Wnt/β-catenin. Latrunculin B (compound 20) and compound 11 strongly inhibited Wnt/β-catenin signaling, a key pathway in the proliferation of epithelial cancer cells. These two compounds affected both alkaline phosphatase and β-catenin levels in C2C12 cells. Compound 11 was the more potent of the two, with an IC₅₀ value of 0.007 μM for β-catenin, and 0.004 μM for alkaline phosphatase.

Effects on Angiogenesis. Angiogenesis inhibition assays were conducted using endothelial colony forming cells (ECFCs). The microvessel density marker CD31 was quantified using confocal laser scanning microscopy and immunohistochemistry. Compounds 11, 14, and 20 all down-regulated the microvessel density marker CD31 in the ECFCs. Each of these three compounds caused 100% inhibition of CD31 in ECFCs at concentrations of 10 μM and 2 μM. The IC₅₀ values for compounds 14 and 20 were 0.063 and 0.043 μM, respectively. The IC₅₀ value for compound 11 was not precisely quantified, but must be below the lowest tested level, 0.043 μM.

Pyrene-actin polymerization assays. Actin polymerization inhibitory activity is assessed using the Cytoskeleton Actin Polymerization kit (Cytoskeleton, Denver, Colo.). Briefly, 5 mM (final concentration) of monomeric actin (1:10 pyrene labeled) is incubated on ice for 10 min with one of various concentrations of the test latrunculin compound. Samples are equilibrated 10 min in an ELISA plate reader (BioTek, VT), after which polymerization is induced by adding KCl, MgCl₂, and ATP. Compounds are tested at 0.1, 0.5, 1.0, and 10 μM. The IC₅₀ for each compound is calculated using GraphPad Prism 5.0.

Cell culture assays. The antiproliferative activity of the latrunculins is measured in vitro using various cell culture assays. Breast cancer cell lines MCF7 and MDA-MB-231, and normal mammary epithelial cells MCF10A (controls) are obtained from ATCC. The cell lines are grown in 10% fetal bovine serum (FBS) (GIBCO-Invitrogen, NY) and Leibovitz's L-15 (ATCC) or RPMI, supplemented with 2 mmol/L glutamine, 100 μg/mL penicillin G, and 100 μg/mL streptomycin, at 37° C. under 5% CO₂.

MTT proliferation assay. The effect of latrunculins on the growth of MCF7 and MDA-MB-231 cancer cell lines, and on MCF10A normal mammary epithelial cells is measured using an MTT kit (TACS™, TREVIGEN®, Inc.). Exponentially-growing cells are plated in a 96-well plate at a density of 8×10³ cells per well, and allowed to attach for 24 hours. Complete growth medium is then replaced with 100 μL of RPMI serum-free medium (GIBCO-Invitrogen, NY) containing one of various doses (50.0, 10.0, 1.0, 0.5, 0.1 μM) of the test latrunculin compound. Culture continues at 37° C. under 5% CO₂. After 72 h of culturing, the cells are treated with MTT solution (10 μL/well) at 37° C. for 4 hours. The color reaction is stopped by adding the solubilization/stop solution (100 μL/well). Incubation continues at 37° C. until complete dissolution of the formazan product. Absorbance of the samples is determined at 570 nm with an ELISA plate reader (BioTek, VT). The number of cells per well is calculated against a standard curve prepared at the start of each experiment by plating various concentrations of cells, as determined by hemocytometer. IC₅₀ for each compound is calculated using non-linear regression of log concentration versus number of cells/well, implemented in GraphPad Prism 5.0.

Cultrex® cell invasion assay. Anti-invasive activities are measured using Trevigen's Cultrex® Cell Invasion Assay. 50 μL of basement membrane extract (BME) coat is added to each well. After incubation for 4 h at 37° C. in 5% CO₂, MDA-50,000 MB-231 cells in 50 μL of serum-free RPMI or Leibovitz's L-15 medium are added to the top chamber of each well with the test compound at one of various concentrations (0.1, 0.5, 1 μM). 150 μL of RPMI medium is added to lower chamber, which contains 10% FBS and penicillin/streptomycin, and which uses fibronectin (1 μL/mL) and N-formyl-met-leu-phe (10 nM) as chemoattractants. Cells are allowed to migrate to the lower chamber at 37° C. under CO₂. After 24 h, the top and bottom chambers are aspirated and washed with the washing buffer supplied with the kit. About 100 μL of cell dissociation solution/calcein-AM solution is added to the bottom chamber and incubated at 37° C. under CO₂ for 1 hour. The cells internalize calcein-AM, and intracellular esterases cleave the acetomethylester (AM) moiety to generate free calcein. Fluorescence measurements of the samples are taken at 485 nm excitation, 520 nm emission using an ELISA plate reader (BioTek, VT). The numbers of cells that invade through the BME coat are calculated using a standard curve.

Wound-healing assay. MDA-MB-231 cells are plated onto sterile 24-well plates and allowed to form a confluent cell monolayer (>95% confluence) overnight. Wounds are then inflicted on each cell monolayer using a sterile 200 μL pipette tip. Media are removed, and cells are washed twice with PBS and once with fresh serum-free media. Test latrunculin compounds at various concentrations (100 nM-10 μM) in fresh, serum-free media are added to each well and incubated for 24 h under serum-starved conditions. Media are then removed, and the cells are washed, fixed and stained using Diff-Quick™ staining (Dade Behring Diagnostics, Aguada, Puerto Rico). Cells that have migrated across the inflicted wound are counted under the microscope in three or more randomly selected fields (magnification: 400×). All experiments are conducted independently in triplicate replications.

Evaluation of Antiproliferative and Antimetastatic Activities of Latrunculins in BALB/c Athymic Nude Mice.

We have demonstrated that the novel latrunculin derivatives significantly inhibit the growth and viability of MDA-MB-231 cells (ATCC-HTB-26) in vitro. The MDA-MB-231 cell line is known to be highly malignant, to be estrogen-independent, and to display anchorage-independent growth when cultured in soft agarose gels. When MDA-MB-231 cells are injected subcutaneously into the flanks of female BALB/c athymic nude mice, they form rapidly-growing, anaplastic adenocarcinomas that are highly invasive, and that typically metastasize into the lungs and bones. The most active latrunculin analogues from the in vitro assays are examined for their antiproliferative and anti-invasive activity in vivo. MDA-MB-231 cells transfected with green fluorescence protein (GFP) gene are grown in culture, isolated with 0.25% trypsin and 0.53 mM EDTA solution, washed, counted and diluted to a desired concentration in fresh, complete growth culture medium. Briefly, 1×10⁶ MDA-MB-231-GFP cells in 100 pL of serum-free RPMI 1640 are injected subcutaneously into the flanks of nude mice using a 1-mL syringe with a 26-gauge, sterile needle. All surgical operations are performed under aseptic conditions. Nude mice with similar tumor sizes, about 100 mm³ (2 weeks post-inoculation) are selected and randomly divided into seven groups (n=9 per group): control (DMSO vehicle); two groups for the two most antiproliferative latrunculins; two groups for the two most anti-invasive latrunculins; and one group for each of latrunculins A and B. Animal weights and tumor growth and metastases are monitored weekly. Average tumor diameter for each palpable tumor is determined as the mean of the two largest perpendicular diameters, measured by vernier calipers. Four to six weeks after tumor cell implantation, tumors in all mice reach approximately 1 cm in diameter. Detection and quantification of primary and metastatic tumors in anesthetized animals in each treatment group are performed with the Kodak In-Vivo Imaging System FX Pro Imaging System. Animals are then given intraperitoneal injections daily for one week, either with vehicle (DMSO) or with the test latrunculin compound. A dose of the test compound equivalent to 0.05 mg per kg body mass is used. (This dose was chosen based on previous reports with latrunculin A. If the dose should be too toxic, an alternative effective dose (middle dose) can be estimated by extrapolation from results obtained from the in vitro studies. A lower dose (½ middle dose) and a high dose (2× middle dose) can then be tested. The doses for each compound can also be titrated to the maximum tolerated doses that do not produce apparent toxicity.)

Afterwards, primary and metastatic tumors in each animal are re-scanned with the same imaging system to determine treatment effects on primary and metastatic tumor growth. Animals are examined every three days for evidence of tumors by fluoroimaging, and the rate of tumor growth is monitored by estimating tumor volume. Animals are examined for secondary tumors in multiple organs by observing the presence of fluorescent cell colonies. At the end of the experimental period, animals are sacrificed. Tumors are excised, weighed, and prepared for later histological examination. Wet sections of organs are examined for the presence of green fluorescent protein. Other tissue portions are fixed in neutral buffered formalin and embedded in paraffin. 5.0 μm-thick sections of tumors are processed for H&E staining. Testing activity against established tumors provides a different (and generally more stringent) test of in vivo anti-tumor activity than the ability to inhibit the formation of new tumors. These experiments determine whether latrunculins can prevent or significantly delay tumor formation and metastasis in vivo. All animal experiments will be approved by the applicable Institutional Animal Care and Use Committee. All surgical and treatment procedures will be consistent with the IACUC policies and procedures, and applicable laws and regulations. Following successful animal testing, clinical trials in humans will be conducted in accordance with applicable laws and regulations.

Statistical Analysis. All experimental treatments are run at least in triplicate. One-way ANOVA is used to evaluate results statistically. Significance is determined by the Newman-Keuls test. Differences are considered statistically significant at p<0.05.

Compounds used in the present invention may be administered to a patient by any suitable means, including intravenous, parenteral, subcutaneous, intrapulmonary, and intranasal administration. Parenteral infusions include intramuscular, intravenous, intraarterial, or intraperitoneal administration. The compounds may also be administered transdermally, for example in the form of a slow-release subcutaneous implant. They may also be administered by inhalation.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampoule, each containing a unit dose amount, or in the form of a container containing multiple doses.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in nanoparticles or microcapsules by techniques otherwise known in the art including, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

As used herein, an “effective amount” of a compound is an amount, that when administered to a patient (whether as a single dose or as a time course of treatment) inhibits or reduces the growth of targeted tumors to a clinically significant degree; or alternatively, to a statistically significant degree as compared to control. “Statistical significance” means significance at the P<0.05 level, or such other measure of statistical significance as would be used by those of skill in the art of biomedical statistics in the context of a particular type of treatment.

The complete disclosures of all references cited in this specification are hereby incorporated by reference. Also incorporated by reference are the complete disclosures of the two priority applications: provisional patent application Ser. No. 61/267,575, and provisional patent application Ser. No. 61/417,942; as well as the complete disclosures of all references cited in either of the two priority applications. In the event of an otherwise irreconcilable conflict, however, the present specification shall control.

### REFERENCES

[1] a) D. J. Newman, G. M. Crag, S. Holbeck, E. A Sausville, Curr. Cancer Drug Targets 2002, 2, 279-308; b) J. R. Peterson, T. J. Mitchison. Chem. & Biol. 2002, 9, 1275-1285; c) G. Fenteany, S. Zhu, Curr. Topics Med. Chem. 2003, 3, 593-616.
[2] a) Y. Kashman, A. Groweiss, U. Shmueli, Tetrahedron Lett. 1980, 21, 3629-3632; b) A. Groweiss, U. Shmueli, Y. Kashman. Marine Toxins of Latrunculia magnifica. J. Org. Chem. 1983, 48, 3512-3516.
[3] Y. Kashman, A. Groweiss, R. Lidor, D. Blasberger, S. Carmely, Tetrahedron 1985, 41, 1905-1914.
[4] K. A. El Sayed, D. T. A Youssef, D. Marchetti, 2006, 69, 219-223.
[5] K. A. El Sayed, M. A. Khanfar, H. M. Shallal, A. Muralidharan, B. Awate, D. T. A. Youssef, Y. Liu, Y. D. Zhou, D. G. Nagle, S. Shah, J. Nat. Prod. 2008, 71, 396-402.
[6] M. Coue, S. L. Brenner, I. Spector, E. D. Korn, FEBS Lett. 1987, 213, 316-318.
[7] C. Hayot, O. Debeir, P. van Ham, M. van Damme, R. Kiss, C. Decaestecker, Toxicol. Appl. Pharmacol. 2006, 211, 30-40.
[8] M. Okka, B. Tian, P. L. Kaufman, Arch. Ophthalmol. 2004, 122, 1482-1488.
[9] P. L. Kaufman, B. Geiger, U.S. Pat. No. 772,412,2002.
[10] I. Spector, N. R. Shochet, D. Blasberger, Y. Kashman, Cell Motil Cytoskel. 1989, 13, 127-144.
[11] C. Hayot, S. Farinelle, R. De Decker, C. Decaestecker, F. Darro, R. Kiss, M. van Damme, Int. J. Oncol. 2002, 21, 417-425.
[12] Y. Gashet, S. Tournier, J. A. B. Millar, J. S. A. Hyams, Nature 2001, 412, 352-355.
[13] W. M. Morton, K. R. Ayscough, P. J. McLaughlin, Nat. Cell Biol. 2000, 2, 376-378.
[14] M. R. Bubb, L. Govindasamy, E. G. Yarmola, S. M. Vorobiev, S.C. Almo, T. Somasundaram, M. S. Chapman, M. Agbandje-McKenna, R. McKenna, J. Biol. Chem. 2002, 277, 20999-21006.
[15] T. Amagata, T. A. Johnson, R. H. Cichewicz, K. Tenney, S. L. Mooberry, J. Media, M. Edelstein, F. A. Valeriote, P. Crews, J. Med. Chem. 2008, 51, 7234-7242.
[16] A. Fürstner, D. Kirk, M. Fenster, C. Aïssa, D. De Souza, C. Nevado, T. Tuttle, W. Thiel, O. Müller, Chem. A E. J. 2006, 13, 135-149.
[17] D. Blasberger, S. Carmely, M. Cojocaru, I. Spector, N. R. Shochet, Y. Kashman, Liebigs Ann. Chem. 1989, 12, 1171-1188.
[18] J. A. Cooper, T. D. Pollard, 1982, 85, 182-211.
[19] L. Zhai, P. Zhao, A. Panebra, A. L. Guerrerio, S. Khurana, J. Biol. Chem. 2001, 276, 36163-36167.
[20] T. Mosmann, J. Immunol. Methods. 1983, 65, 55-63.
[21] M. C. Alley, D. A. Scudiero, A. Monks, M. L. Hursey, M. J. Czerwinski, D. L. Fine, B. J. Abbott, J. G. Mayo, R. H. Shoemaker, M. R. Boyd, Cancer Res. 1988, 48, 589-601.
[22] K. P. Tamilarasan, G. K. Kolluru, M. Rajaram, M. Indhumathy, R. Saranya, S. Chatterjee, BMC Cell Biol. 2006, 7, 17-30.
[23] P. R. Borghesani, J. M. Peyrin, R. Klein, J. Rubin, A. R. Carter, P. M. Schwartz, A. Luster, G. Corfas, R. A. Segal, Development. 2002, 6, 1435-1442.
[24] Tripos Associates. SYBYL Molecular Modeling Software, version 8.0; Tripos Associates: St. Louis, Mo., 2007; http://www.tripos.com.
[25] M. Clark, R. D. III Cramer, N. van Opdenbosch, Comput. Chem. 1989, 10, 982-1012.
[26] J. J. Stewart, J. Comput. Aided Mol. Des. 1990, 4, 1-105.
[27] E. Kellenberger, J. Rodrigo, P. Muller, D. Rognan, Proteins, 2004, 57, 225-242.
[28] A. N. Jain, J. Med. Chem. 2003, 46, 499-511.
[29] W. Welch, J. Ruppert, A. N. Jain, Chem. Biol. 1996, 3, 449-462.
[30] J. Ruppert, W. Welch, A. N. Jain, Protein Sci. 1997, 6, 524-533.
[31] Khanfar M A, Youssef DTA, El Sayed K A. Semisynthetic latrunculin derivatives as inhibitors for metastatic breast cancer: Biological evaluations, preliminary structure-activity relationship, and molecular modeling studies. ChemMedChem, 2010, 5, 274-285.
[32] Khanfar M A, El Sayed K A. 3D-QSAR Studies of latrunculin-based actin polymerization inhibitors using CoMFA and CoMSIA approaches. European J Med. Chem., 2010, 9, 3662-3668.

We claim:

1. A compound having structure A, B, or C as shown: 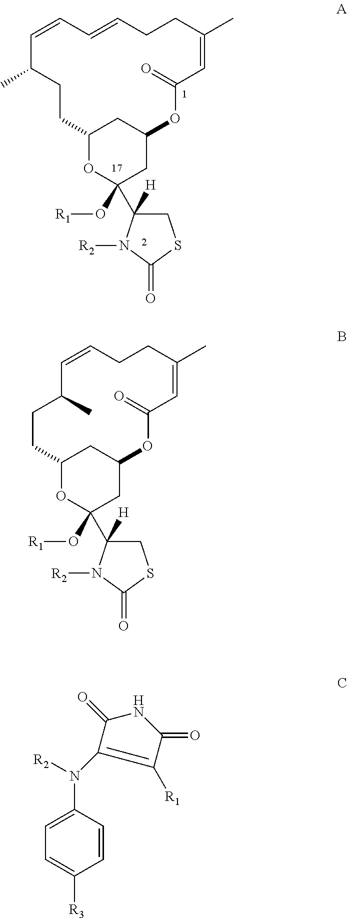
wherein R₁, R₂, and R₃ may be the same or different; and wherein each of R₁, R₂, and R₃ is independently selected from the group consisting of H, CH₃, CH₂CH₃, OH, SH CH₂OH, CHO, COOH, CONH₂, NH₂, C1 to C8 substituted or unsubstituted alkyl, C1 to C8 substituted or unsubstituted hydroxyalkyl, C1 to C8 substituted or unsubstituted alkoxy or hydroxyalkoxy, C1 to C8 substituted or unsubstituted carboxyalkyl, C1 to C8 substituted or unsubstituted carbonylalkyl, substituted or unsubstituted aryl or heteroaryl, substituted or unsubstituted hydroxyaryl or hydroxyheteroaryl, C1 to C8 substituted or unsubstituted amino or amide; N-hydroxylamine; C1 to C8 substituted or unsubstituted oxime;
provided that:
if the compound has structure A and R₁ is H, then R₂ is neither H nor CH₂OH;
and further provided that:
if the compound has structure B, then R₁ and R₂ are not both H.

2. The compound of claim 1, wherein said compound has structure A; R₁ is CH₃; and R₂ is H.

3. The compound of claim 1, wherein said compound has structure A; R₂ is H; and R₁ is

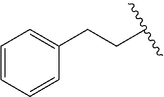

4. The compound of claim 1, wherein said compound has structure A; R₂ is H; and R₁ is

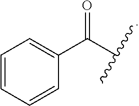

5. The compound of claim 1, wherein said compound has structure B; R₁ is CH₃; and R₂ is

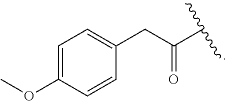

6. The compound of claim 1, wherein said compound has structure A; R₁ is CH₃; and R₂ is CH₂NOH.

7. The compound of claim 1, wherein said compound has structure A; R₁ is CH₃; and R₂ is

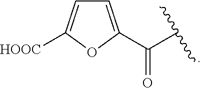

8. The compound of claim 1, wherein said compound has structure A; R₁ is CH₃; R₂ is (CH₂)ₙOH; and n is 1, 2, or 3.

9. The compound of claim 1, wherein said compound has structure A; R₁ is CH₃; and R₂ is

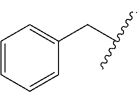

10. The compound of claim 1, wherein said compound has structure A; R₁ is CH₃; and R₂ is

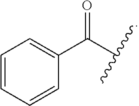

11. The compound of claim 1, wherein said compound has structure A; R₁ is CH₃; and R₂ is

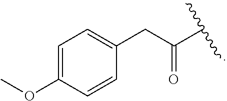

12. The compound of claim 1, wherein said compound has structure A; R₁ is H; and R₂ is CH₂NOH.

13. The compound of claim 1, wherein said compound has structure A; R₁ is H; and R₂ is

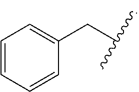

14. The compound of claim 1, wherein said compound has structure A; R₁ is CONHC₆H₅; and R₂ is H.

15. The compound of claim 1, wherein said compound has structure A; R₁ is CONHCH₂C₆H₅; and R₂ is H.

16. The compound of claim 1, wherein said compound has structure A; R₂ is CH₂OH; and R₁ is

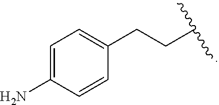

17. The compound of claim 1, wherein said compound has structure A; R₂ is CH₂OH; and R₁ is

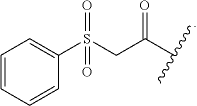

18. The compound of claim 1, wherein said compound has structure A; R₁ is H; and R₂ is

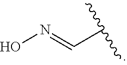

19. The compound of claim 1, wherein said compound has structure A; R₂ is CH₂OH; and R₁ is

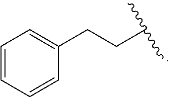

20. The compound of claim 1, wherein said compound has structure A; R₁ is (CH₂)₄COOH; and R₂ is H or CH₂OH.

21. The compound of claim 1, wherein said compound has structure A; R₂ is H or CH₂OH; and R₁ is

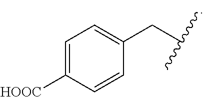

22. The compound of claim 1, wherein said compound has structure A; R₁ is H; and R₂ is CH₂NH₂.

23. The compound of claim 1, wherein said compound has structure A; R₁ is H; and R₂ is

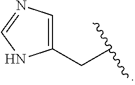

24. The compound of claim 1, wherein said compound has structure B; R₁ is H; and R₂ is COCH₃.

25. The compound of claim 1, wherein said compound has structure B; R₁ is CH₃; and R₂ is

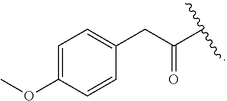

26. The compound of claim 1, wherein said compound has structure B; R₁ is H; and R₂ is CH₂OH.

27. A method for killing or inhibiting the growth of cells in a tumor in a mammal; said method comprising administering to the mammal an effective amount of the compound of claim 1.

28. The method of claim 27, wherein the tumor is selected from the group consisting of prostate, breast, ovary, urothelial, pancreas, glioblastoma, melanoma, ocular melanoma, gastric, and non-small cell lung cancers.

29. The method of claim 27, wherein the compound has structure A; R₁ is CH₃; and R₂ is [chemical structure]
